(19) United States
(12) Patent Application Publication
(10) Pub. No.: US 2026/0263225 A1
(43) Pub. Date: Sep. 10, 2026

(54) IMPROVEMENTS IN AND RELATING TO JOINT PROSTHESES, KITS AND METHODS OF USE

(71) Applicant: DEPUY IRELAND UNLIMITED COMPANY, Ringaskiddy Co. Cork (IE)

(72) Inventors: Colin DUDREY-HARVEY, Leeds (GB); David HORNE, Leeds (GB); Jonathan MEW, Leeds (GB)

(21) Appl. No.: 19/469,206

(22) PCT Filed: Mar. 27, 2024
(86) PCT No.: PCT/EP2024/058354

§ 371 Date: Sep. 25, 2025

(30) Foreign Application Priority Data
Apr. 3, 2023 (GB) 2304905.9

(51) Int. Cl. A61F 2/34; A61F 2/46

(52) U.S. Cl. CPC A61F 2/34; A61F 2/4637; A61F 2002/3401

(57) ABSTRACT

An acetabular component of an orthopaedic joint prosthesis which has an outer shell for mounting on the acetabulum; a liner provided at least partially within the outer shell for providing a contact surface for a femoral component of the orthopaedic joint prosthesis; and an adapter provided at least partially within the outer shell, where the liner is at least partially within the adapter. The adapter allows a wider range of liners to be used with a given shell.

### TECHNICAL FIELD

This disclosure concerns improvements in and relating to joint prostheses, kits including those and methods of using those. The disclosure is particularly, but not exclusively, concerned with orthopaedic joint prostheses such as the acetabular part thereof.

### BACKGROUND

In orthopaedic uses, a variety of different acetabular cup replacement approaches can be taken. Only a few of these are suitable to be used in complex acetabular defect surgery where more traditional shells with fixings that engage the bone cannot be used successfully. Fully porous shells with cemented in liners provide one such approach. However, the fully porous shell is designed to be complimentary to a given size and type of liner that is cemented to it. Thus, the liner size and position are constrained for any given shell and so such an approach is unable to offer as great a range of sizes and positions for the implant as modular systems. It would be beneficial to increase the range of sizes and positions possible, potentially through the use of non-cemented liners.

### SUMMARY

According to a first aspect of the invention there is provided a component of a prosthesis, the component comprising:
an outer shell;
a liner provided at least partially within the outer shell and adapted to provide a contact surface for a further component of the prosthesis; and
an adaptor provided at least partially within the outer shell and with the liner provided at least partially within the adaptor.

The component may further provide that the acetabular component has an assembled state and in the assembled state relative articulation between the adaptor and the outer shell is prevented. The component may further provide that the acetabular component has an assembled state and in the assembled state relative articulation between the adapter and the liner is prevented. The component may further provide that the acetabular component has an assembled state and in the assembled state relative articulation between the adaptor and the outer shell is prevented and in the assembled state relative articulation between the adapter and the liner is prevented.

The component may be an acetabular component. The component may be a component of an orthopaedic joint prosthesis. The outer shell may be for mounting on the acetabulum.

The further component may be a femoral component, for instance of an orthopaedic joint prosthesis.

The adapter may have an engaged state with the shell. The adapter may be at least partially within the shell in the engaged state.

In the engaged state of the shell and adapter, a plurality of attachment elements may engage with a plurality of attachment locations. At least one of attachment elements and/or attachment locations may be provided on the adapter, for instance with the other being provided on the shell.

All of the attachment elements may be provided on the adaptor. All of the attachment locations may be provided on the shell. Alternatively, all of the attachment elements may be provided on the shell and/or all of the attachment locations may be provided on the adapter. Each attachment element may match to an attachment location. The number of attachment locations may match or exceed the number of attachment elements.

The adapter may have a disengaged state and an engaged state with the shell. The adapter may be wholly outside of the shelll in the disengaged state of the shell and adapter.

In the disengaged state of the shell and adapter, a plurality of attachment elements may be disengaged from a plurality of attachment locations. At least one of attachment elements and/or attachment locations may be provided on the adapter, for instance with the other being provided on the shell.

The adapter may also have a transition state between the disengaged state and the engaged state of the shell and adapter. The adapter may have a first configuration in the transition state and a different second configuration in the engaged state.

The adapter may have a first configuration in the disengaged state of the shell and adapter. The attachment elements or attachment locations provided on the adapter may have a greater circumferential and/or radial extent in the first configuration, than in a second configuration and/or than in a third configuration. The attachment elements or attachment locations provided on the adapter may have a maximum circumferential and/or radial extent in the first configuration.

The adapter may have a second configuration in a transition state. The adapter may have a third configuration in a transition state.

During or in the transition state of the shell and adapter, the circumferential extent and/or radial extent of the attachment elements and/or attachment locations may decrease, for instance to a minimum. In the engaged state of the shell and adapter, the circumferential extent and/or radial extent of the attachment elements and/or attachment locations may be below the maximum and/or above the minimum. The circumferential and/or radial extent of the attachment elements and/or attachment locations may decrease from a maximum to a lower extent as the attachment elements enter the attachment locations.

The liner may have an engaged state with the adapter. The liner may be at least partially within the adapter in the engaged state.

In the engaged state of the liner and adapter, a plurality of abutment elements and/or abutment locations may abut with a plurality of abutment locations and/or abutment elements. At least one of abutment elements and/or abutment locations may be provided on the adapter, for instance with the other being provided on the liner.

All of the abutment elements may be provided on the adaptor. All of the abutment locations may be provided on the liner. Alternatively, all of the abutment elements may be provided on the liner and/or all of the abutment locations may be provided on the adapter. Each abutment element may match to an abutment location. The number of abutment locations may match or exceed the number of abutment elements.

The adapter may have a disengaged state and an engaged state with the liner. The liner may be wholly outside of the adapter in the disengaged state of the liner and adapter.

In the disengaged state of the liner and adapter, a plurality of abutment elements may be disengaged from a plurality of abutment locations. At least one of abutment elements and/or abutment locations may be provided on the adapter, for instance with the other being provided on the liner.

The liner may also have a transition state between the disengaged state and the engaged state of the liner and adapter. The liner may have a first configuration in the transition state and a different second configuration in the engaged state.

The liner may have a first configuration in the disengaged state of the liner and adapter. The liner may have a maximum radial extent in the disengaged state of the liner and adapter. The liner may have a second configuration in a transition state. The liner may have a third configuration in a transition state. The liner may have the maximum radial extent in the transition state of the liner and adapter. The liner may have a reduced radial extent in the transition state of the liner and adapter, for instance due to compression of the liner by the adapter. The liner may have the maximum radial extent in the engaged state of the liner and adapter. The liner may have a reduced radial extent in the engaged state of the liner and adapter, for instance due to compression of the liner by the adapter.

The outer shell may abutted by the adapter and/or the adapter is abutted by the outer shell and/or the adapter is abutted by the liner and/or the liner is abutted by the adapter, when the component of the implant is in an assembled state. The abutment may prevent relative movement of the adapter and shell and/or liner and adapter. The outer shell may be compressed by the adapter and/or the adapter is compressed by the outer shell and/or the adapter is compressed by the liner and/or the liner is compressed by the adapter, when the component of the implant is in an assembled state. The compression may prevent relative movement of the adapter and shell and/or liner and adapter.

The component of the implant may have a disassembled state and/or a partially assembled state and/or an assembled state.

The component of the implant may have a disassembled state wherein the shell and adapter are in a disengaged state and wherein the adapter and liner are in a disengaged state. The component of the implant may have a partially assembled state wherein one of the shell and adapter or adapter and liner are in a disengaged state and wherein the other of the shell and adapter or adapter and liner are in a disengaged state. The component of the implant may have an assembled state wherein the shell and adapter are in an engaged state and wherein the adapter and liner are in an engaged state.

In the implant component assembled state, the liner may have one or more abutment elements and/or abutment locations which abut the adapter. The abutment elements and/or location may prevent relative movement of the liner to another part, such as the outer shell and/or the adapter. In the implant component assembled state, the adapter may have one or more abutment elements and/or abutment locations which abut the liner. The abutment elements and/or location may prevent relative movement of the adapter to another part, such as the outer shell and/or the liner. In the implant component assembled state, the adapter may have one or more attachment locations and/or attachment elements which engage the shell. The abutment elements and/or location may prevent relative movement of the adapter to another part, such as the outer shell and/or the liner. In the implant component assembled state, the shell may have one or more attachment locations and/or attachment elements which engage the adapter. The abutment elements and/or location may prevent relative movement of the outer shell to another part, such as the liner and/or the adapter.

In the implant component assembled state, the liner may have a reduced configuration compared with the partially assembled state and/or disassembled state. The reduced configuration may be due to one or more compressed parts of the liner, for instance compressed by the adapter, for instance compressed radially inwardly. The reduced configuration may be provided evenly around the perimeter of the liner or may be localised. The reduced configuration may prevent relative movement of the liner and/or the outer shell and/or the adapter.

In the implant component assembled state, the adapter may have a reduced configuration compared with the partially assembled state and/or disassembled state. The reduced configuration may be due to one or more compressed parts of the adapter, for instance compressed by the shell, for instance compressed radially inwardly. The reduced configuration may be provided evenly around the outer perimeter of the adapter or may be localised. The reduced configuration may prevent relative movement of the liner and/or the outer shell and/or the adapter.

In the implant component assembled state, the adapter may have an increased dimension compared with the partially assembled state and/or disassembled state. The increased dimension may be due to one or more compressed parts of the adapter, for instance compressed by the liner, for instance compressed radially outwardly. The increased dimension may be provided evenly around the inner perimeter of the adapter or may be localised. The increased dimension may prevent relative movement of the liner and/or the outer shell and/or the adapter.

In the implant component assembled state, the shell may have an increased dimension compared with the partially assembled state and/or disassembled state. The increased dimension may be due to one or more compressed parts of the shell, for instance compressed by the adapter, for instance compressed radially outwardly. The increased dimension may be provided evenly around the inner perimeter of the shell or may be localised. The increased dimension may prevent relative movement of the liner and/or the outer shell and/or the adapter.

Particularly in a first general embodiment, the following features or options may be provided.

The adaptor may include a body element. The adaptor may include a unitary body element, for instance extending from a base section to a perimeter section. The adaptor may include outer shell connection elements and/or outer shell connection locations being provided on or in the perimeter section.

The body element may define a recess, for instance to receive at least a part of the liner. The recess may be provided in the distal surface of the adapter. The recess may be a through aperture. The recess may be a cup or other closed shape.

The recess may be provided with a defined size and/or defined diameter and/or defined depth and/or defined position within the adapter and/or a defined recess internal profile to receive one or more liners. The recess may be so provided to receive a liner selected from a plurality of liners which have one or more different characteristics.

The one or more different characteristics between liners may be selected from the group comprising: one or more distal characteristics; one or more distal surface characteristics; one or more different sizes; one or more different defined positions for the contact surface of the liner; one or more different contact surface profiles.

The one or more different characteristics between liners, the liners having a cup defining the contact surface, may be selected from the group comprising: the cup size; the cup diameter; the cup depth; the cup position within the liner; the cup internal profile.

The outer shell connection elements and/or outer shell connection locations provided on the adapter may be provided in or on the perimeter section of the adapter. The perimeter section of the adaptor may be provided by one or more flanges extending from the body element. The flange may have an even outward extend around the adapter. The flange may have an uneven outward extent around one or more parts of the adapter. A single flange may be provided. The flange may have a distal surface and a proximal surface. The outer shell connection elements and/or outer shell connection locations be provided on the proximal surface of the flange.

The outer shell connection elements and/or outer shell connection locations may be provided at two or more, three or more or four or more locations. The outer shell connection elements and/or outer shell connection locations may be regularly and/or evenly spaced around the perimeter section.

In an assembled state, the proximal perimeter surface of the adapter may be aligned with and particularly may abut with a distal surface of the outer shell. In the assembled state, one or more outer shell connection elements and/or outer shell connection locations on the adapter may engage with one or more outer shell connection locations and/or outer shell connection elements on the liner. This abutting may prevent relative movement of the liner and/or the outer shell and/or the adapter.

One or more parts of one or more or all of the outer shell connection elements and/or outer shell connection locations may be deformable.

One or more or all of the outer shell connection elements, for instance provided on the adapter, may be profiled with one or more inclined side surfaces. One or more or all of the outer shell connection elements may include an end surface, for instance a proximal end surface when provided on the adapter. One or more or all of the outer shell connection elements may be provided with a neck location, for instance at the junction with the adapter and/or outer shell. One or more or all of these surfaces and/or neck locations may be complimentary to those of the outer shell connection locations provided on the other of the adapter and/or outer shell.

One or more or all of the outer shell connection elements may be in the form of a pair of fingers extending from the adapter and/or outer shell. One or both of the fingers may be inclined relative to the surface they extend from, particularly in opposing directions of inclination. A deformation permitting space may be provided between the fingers. The fingers may be deformable.

The liner may have a reduced radial extent in the engaged state of the liner and adapter, for instance due to compression of the liner by the adapter.

Particularly in a second general embodiment, the following features or options may be provided.

The adaptor may include a base element.

The adaptor may include one or more shell attachment elements and/or attachment locations being provided on base element. The adapter may include one or more liner abutment elements and/or one or more liner abutment locations provided on one or more arm elements.

The adaptor may include a plurality of arm elements, for instance extending from a base element.

One or more or all of the arm elements may include a base section, for instance connecting to the base element. One or more or all of the arm elements may include an end section, for instance a distal end section. One or more or all of the arm elements may include abutment elements and/or abutment locations provided in the end section. One or more or all of the arm elements may include an intermediate section. The intermediate section may be provided connected to the base section and/or to the end section.

One or more or all of the arm elements may be curved. One or more or all of the arm elements may have one or more curved sections. One or more or all of the arm elements may have a shape complementary to the shape of the adjacent part, in the assembled state, of the outer shell. One or more or all of the arm elements may have a shape complementary to the shape of the adjacent part, in the assembled state, of the liner.

One or more or all of the arm elements may have an external surface complementary, for instance at least in part, to the internal surface of the adjacent part, in the assembled state, of the outer shell. One or more or all of the arm elements may have an internal surface complementary, for instance at least in part, to the external surface of the adjacent part, in the assembled state, of the liner.

The arm elements of the adaptor may define a substantially hemispherical profile.

The arm elements may be flexible.

The arm elements may flex radially further outward in the transition state compared with the assembled state. The intermediate section of the arm element may provide for the flexing of the arm element and/or the base section of the arm element may provide for the flexing of the arm element. The end section may have a degree of flexibility.

The adapter may include at least two arm elements. The adapter may include at least three arm elements. The adapter may include at least four arm elements. The adapter may include at least two arm elements. The arm elements may be regularly spaced around the adapter. The arm elements may be provided in opposing pairs.

One or more or all of the arm elements may be finger-shaped, for instance a curved finger. One or more or all of the arm elements may be petal-shaped, for instance a curved petal.

The abutment elements and/or abutment locations provided on the adapter may be provided at the distal periphery of the adapter. The abutment elements and/or abutment locations may extend around at least 10% of the distal periphery of the adapter. The abutment elements and/or abutment locations may extend around at least 15% of the distal periphery of the adapter. The abutment elements and/or abutment locations may extend around at least 50% of the distal periphery of the adapter. The abutment elements and/or abutment locations may extend around at least 80% of the distal periphery of the adapter.

The abutment elements and/or abutment locations provided on the adapter may form the distal periphery of the adapter. The abutment elements and/or abutment locations may extend around the entire distal periphery of the adapter, such as with adjacent abutment elements and/or abutment locations abutting or overlapping with one another.

The adapter may be provided with one or more abutment elements and/or abutment locations and the abutment elements and/or abutment locations may be abutted by the outer shell and/or by the liner. The adapter may be provided with one or more abutment elements and/or abutment locations which are pressed or compressed between an adjacent part of the outer shell and an adjacent part of the liner.

The adapter may be provided with one or more abutment elements and/or abutment locations which are pressed or compressed between one or more abutment locations and/or abutment elements on an adjacent part of the outer shell and/or between one or more abutment locations and/or abutment elements on an adjacent part of the liner.

The abutment elements of the adapter may be pressed or compressed into the attachment locations of the shell and/or of the liner. The abutment locations of the adapter may receive attachment elements of the shell and/or of the liner.

The liner may have a reduced radial extent in the engaged state of the liner and adapter, for instance due to compression of the liner by the adapter.

All the general embodiments of the disclosure may include the following features and options.

The adapter may have an outer shell disengaged state and an outer shell engaged state with the outer shell. The adapter may be wholly outside of the outer shell in the disengaged state. The adapter may also have an outer shell transition state between the outer shell disengaged state and the outer shell engaged state.

Particularly in a first general embodiment, the following features or options may be provided.

The adapter may include a projection from the base element, for instance proximally. The adaptor may provide the one or more shell attachment elements and/or attachment locations on the projection. The shell may include an aperture to receive at least a part of the adapter projection. The aperture may be an open or closed aperture. The projection and aperture and their features may be reversed by provision on the shell and the adapter instead.

The outer shell connection elements and/or outer shell connection locations provided on the outer shell may be provided in or on the shell perimeter section of the outer shell. The shell perimeter section of the outer shell may be provided by a shell perimeter face, particularly a proximal face. The shell perimeter face may have an even outward extend around the outer shell. The shell perimeter face may have an uneven outward extent around one or more parts of the outer shell. The outer shell connection elements and/or outer shell connection locations may be provided on the distal shell perimeter face.

The outer shell connection elements and/or outer shell connection locations may be provided at two or more, three or more or four or more locations. The outer shell connection elements and/or outer shell connection locations may be regularly and/or evenly spaced around the shell perimeter section.

In an assembled state, the distal shell perimeter surface of the adapter may be aligned with and particularly may abut with a proximal surface of the adapter. In the assembled state, one or more outer shell connection elements and/or outer shell connection locations on the outer shell may engage with one or more outer shell connection locations and/or outer shell connection elements on the adapter.

One or more parts of one or more or all of the outer shell connection elements and/or outer shell connection locations provided by the outer shell may be deformable.

One or more or all of the outer shell connection elements, for instance provided on the outer shell, may be profiled with one or more inclined side surfaces. One or more or all of the outer shell connection elements may include an inner base surface, for instance a distal facing base surface when provided on the outer shell. One or more or all of the outer shell connection elements may be provided with a neck location or a mouth location, for instance at the junction with the shell perimeter face and/or distal relative to the inner end face. The neck location or mouth location may have a reduced cross-section compared with the base surface cross-section. One or more or all of these surfaces and/or neck locations and/or mouth locations may be complimentary to those of the outer shell connection locations provided on the other of the adapter and/or outer shell.

One or more outer shell connection elements and/or outer shell connection locations may be provided within the recess of the outer shell at one or more recess location. The one or more recess locations may be an aperture, for instance a through aperture. The aperture may be suitable to receive a fixing element, such as a screw. A recess location may be provided at the proximal base of the recess. One or more recess locations may be provided intermediate the proximal base of the recess and the perimeter of the recess.

Particularly in a second general embodiment, the following features or options may be provided.

The adaptor may provide the one or more shell attachment elements as a threaded section on the projection, for instance an external thread on the projection. The shell may provide the one or more attachment locations as a threaded section on the aperture, for instance an internal thread in the aperture. The projection and aperture and their features may be reversed by provision on the shell and the adapter instead.

The outer shell connection element may include a threaded element, for instance on a protruding stem, such as a proximally protruding stem. The adapter connection element may include a threaded element, for instance in an aperture, such as recessed into or extending through a hole in the outer shell. The outer shell connection element may be centrally provided on the adapter, for instance on the external surface. The adapter connection element may be centrally provided on the outer shell, for instance at the base inside the outer shell.

Particularly in a third general embodiment, the following features or options may be provided.

The adaptor may provide the one or more shell attachment elements on the projection which insert into one or more attachment locations in the aperture of the shell. For instance, the projection may include a first cross-sectional part, second reduced cross-section part and a third intermediate cross-sectional part. The third cross-sectional part may be adapted to pass through a reduced cross-section part of the aperture in the shell and pass into an increased cross-section part of the aperture. The third cross-sectional part of the projection and/or the reduced cross-section part of the aperture may be deformable, for instance to allow passage. The projection and aperture and their features may be reversed by provision on the shell and the adapter instead.

The outer shell connection element may include one or more distal facing abutment surfaces. The adapter connection element may include one or more proximal facing abutment surfaces. The one or more distal facing abutment surfaces may abut the one or more proximal facing abutment surfaces, for instance in the assembled state.

The outer shell connection element may comprise a first section of a first cross-section. The outer shell connection element may comprise a second section of a second cross-section. The second section may be proximal to the first section. The first cross-section may be larger, for instance in diameter and/or area, than the second cross-section. The outer shell connection element may comprise a third section of a third cross-section. The third section may be proximal to the first section and/or the second section. The third cross-section may be intermediate, for instance in diameter and/or area, to the second cross-section and/or to the second cross-section. The second cross-section may be smaller in cross-section than the first cross-section and/or than the third cross-section. One or more or all of the cross-sections may be considered perpendicular to the axis of insertion of the adapter into the outer shell.

The third section may provide the one or more distal facing abutment surfaces.

The third section may include one or more flexible elements, such as stems. An axial space may be provided between the flexible elements. The second section may also contribute to or be part of the one or more flexible elements.

The outer shell connection element and the adapter connection element may be complimentary to one another in profile over one or more or all sections.

The adapter connection element may accommodate the entirety of the outer shell connection element of the adapter within the external surface profile of the outer shell. The outer shell connection element may be recessed within the external surface profile of the outer shell in the assembled state.

The adapter connection element may comprise a first shell section of a first shell cross-section. The adapter connection element may comprise a second shell section of a second shell cross-section. The second shell section may be distal to the first shell section. The first shell cross-section may be larger, for instance in diameter and/or area, than the second shell cross-section. The adapter connection element may comprise a third shell section of a third shell cross-section. The third shell section may be distal to the first shell section and/or the second shell section. The third shell cross-section may be larger, for instance in diameter and/or area, than the second shell cross-section and/or the first shell cross-section. The second shell cross-section may be intermediate in cross-section to the first shell cross-section and/or third shell cross-section. One or more or all of the cross-sections may be considered perpendicular to the axis of insertion of the adapter into the outer shell. The third section may provide the one or more distal facing abutment surfaces. The distal facing abutment surfaces of the outer shell connection element of the adapter may be provided on one or more deformable elements, such as stems.

All the general embodiments of the disclosure may include the following features and options.

The outer shell may include one or more sections of porous material. The outer shell may be completely formed of porous material. The outer shell may be porous in terms of mimicking cancellous bone. An outer shell completely formed of porous material may mimic cancellous bone throughout the depth and extend of the outer shell. A unitary outer shell may be provided.

The outer shell may be provided with a curved outer surface, for instance a part or whole of a hemisphere. The outer shell may be provided with a curved inner surface, for instance a part or whole of a hemisphere. The outer shell may be provided with a shell recess, for instance in the distal side. The shell recess may receive at least a part of the adapter. The shell recess may receive at least part of the liner. The shell recess may be provided with a defined size and/or defined diameter and/or defined depth and/or defined position within the outer shell and/or a defined recess internal profile to receive an adapter.

The outer shell may be provided with one or more through apertures, for instance adapted to cooperate with a fixing, such as a screw.

The liner may be provided with a curved inner surface, for instance a part or whole of a hemisphere.

A unitary liner may be provided.

The liner contact surface may provide a bearing surface for another component of the prosthesis.

The first aspect of the disclosure may include any of the features, options or possibilities set out elsewhere in this document, including in the other aspects of the disclosure.

According to a second aspect of the disclosure there is provided a kit of surgical components comprising:
a component of a prosthesis, the component comprising:
an outer shell;
a plurality of liners, at least one of the liners having one or more different characteristics than another one of the liners;
an adaptor;
wherein the plurality of liners are adapted to be provided at least partially within the outer shell and adapted to provide a contact surface for a further component of the prosthesis; and
wherein the adaptor is adapted to be provided at least partially within the outer shell and with one of the liners provided at least partially within the adaptor.

The kit may provide for an acetabular component having an assembled state and in the assembled state relative articulation between the adaptor and the outer shell is prevented. The kit may provide for an acetabular component having an assembled state and in the assembled state relative articulation between the adapter and the liner is prevented. The kit may provide for an acetabular component having an assembled state and in the assembled state relative articulation between the adaptor and the outer shell is prevented and in the assembled state relative articulation between the adapter and the liner is prevented.

The component may be an acetabular component. The component may be a component of an orthopaedic joint prosthesis. The outer shell may be for mounting on the acetabulum. The further component may be a femoral component, for instance of an orthopaedic joint prosthesis.

The one or more different characteristics between liners may be that one or more liners is a cemented type liner and that one or more liners is a non-cemented type liner, particularly non-modified cemented-type liners and non-modified non-cemented type liners.

A non-cemented type liner may be provided with one or more connection locations and/or connection elements. The connection locations and/or connection elements may be discrete parts extending from and/or recess relative to the remainder of the liner. The connection locations and/or connection elements may be pre-existing parts for cooperation with an outer shell, but repurposed for cooperation with the adapter. The connection locations may be abutment locations and/or abutment elements. The connection locations may be pre-existing abutment locations and/or abutment elements for cooperation with abutment elements and/or abutment locations on an outer shell repurposed for cooperation with abutment elements and/or abutment locations of the adapter.

A cemented type liner may be devoid of connection locations and/or connection elements. A cemented type liner may be devoid of connection locations and/or connection elements which are discrete parts extending from and/or recess relative to the remainder of the liner.

The one or more different characteristics between liners may be selected from the group comprising: one or more distal characteristics; one or more distal surface characteristics; one or more different sizes; one or more different defined positions for the contact surface of the liner; one or more different contact surface profiles.

The one or more different characteristics between liners, the liners having a cup defining the contact surface, may be selected from the group comprising: the cup size; the cup diameter; the cup depth; the cup position within the liner; the cup internal profile.

The liners may have the same characteristics in respect of some characteristics, for instance the proximal characteristics.

The kit may further comprise: one or more further outer shells, at least one of the outer shells having one or more different shell characteristics than another one of the outer shells. The kit may further comprise the one or more different characteristics between outer shells is that one or more outer shells is a cemented outer shell and/or one or more outer shells is a non-cemented outer shell.

A non-cemented outer shell may be provided with one or more connection locations and/or connection elements. The connection locations and/or connection elements may be discrete parts extending from and/or recess relative to the remainder of the outer shell. The connection locations and/or connection elements may be pre-existing parts for cooperation with a liner, but repurposed for cooperation with the adapter. The connection locations may be attachment locations and/or attachment elements. The connection locations may be pre-existing attachment locations and/or attachment elements for cooperation with attachment elements and/or attachment locations on a liner repurposed for cooperation with attachment elements and/or attachment locations of the adapter.

A cemented outer shell may be devoid of connection locations and/or connection elements. A cemented outer shell may be devoid of connection locations and/or connection elements which are discrete parts extending from and/or recess relative to the remainder of the outer shell.

A cemented outer shell may be an outer shell adapted for fixing to the patient by cement. A non-cemented outer shell may be an outer shell adapted for fixing to the patent other than by cement, for instance by one or more bone fixings.

A non-cemented shell may be provided with one or more mounting locations, such as fixation mounting locations. The one or more fixation mounting locations may be apertures and/or slots extending through the non-cemented shell. The one or more fixation mounting locations may receive a fixation, in use, such as a screw and/or nail and/or pin. The fixation may be a bone engaging fixation. The one or more fixation mounting locations may be provided with an expanded profile section, for instance to receive a head portion of the fixation, in use.

A cemented shell may be devoid of mounting locations, potentially fixation mounting locations, such as apertures or slots extending through the cemented shell.

The cemented shell may be provided with one or more cement contacting surfaces. The cemented shell may physically and/or chemically interact with cement on contact.

The one or more different outer shell characteristics may be selected from the group comprising: one or more distal characteristics; one or more distal surface characteristics; one or more different sizes; one or more different interior profiles; one or more different internal adapter contact surface profiles; one or more proximal characteristics; one or more proximal surface characteristics; one or more surface coatings or surface materials; one or more materials.

The kit may further comprise: one or more further adapters, at least one of the adapters having one or more different adapter characteristics than another one of the adapters. The one or more different adapter characteristics may be selected from the group comprising: one or more distal characteristics; one or more distal surface characteristics; one or more different sizes; one or more different interior profiles; one or more different internal liner contact surface profiles; one or more proximal characteristics; one or more proximal surface characteristics; one or more different external profiles; one or more different external outer shell contact surface profiles; one or more materials.

The kit may further comprise: one or more femoral components; and/or one or more surgical instruments. The femoral components may comprise one or more stems and/or one of more heads.

The second aspect of the disclosure may include any of the features, options or possibilities set out elsewhere in this document, including in the other aspects of the disclosure.

According to a third aspect of the disclosure there is provided a method of assembling a component of a prosthesis, the method comprising:
obtaining an adaptor, a liner and an outer shell;
inserting the liner into the adapter;
inserting the adapter into the outer shell;
   wherein the component comprises:
an outer shell;
a liner provided at least partially within the outer shell and adapted to provide a contact surface for a further component of the prosthesis; and
an adaptor provided at least partially within the outer shell and with the liner provided at least partially within the adaptor.

The method may further provide the acetabular component in an assembled state and in the assembled state relative articulation between the adaptor and the outer shell is prevented. The method may further provide the acetabular component in an assembled state and in the assembled state relative articulation between the adapter and the liner is prevented. The method may further provide the acetabular component in an assembled state and in the assembled state relative articulation between the adaptor and the outer shell is prevented and in the assembled state relative articulation between the adapter and the liner is prevented.

The method may include the component being an acetabular component. The component may be a component of an orthopaedic joint prosthesis. The outer shell may be for mounting on the acetabulum. The further component may be a femoral component, for instance of an orthopaedic joint prosthesis.

The method may include selecting the liner from amongst a plurality of liners, at least one of the liners having one or more different characteristics than another one of the liners. The one or more different characteristics between liners may be that one or more liners is a cemented liner and that one or more liners is a non-cemented liner, particularly non-modified cemented type liners and non-modified non-cemented type liners.

A non-cemented type liner may be provided with one or more connection locations and/or connection elements. The connection locations and/or connection elements may be discrete parts extending from and/or recess relative to the remainder of the liner. The connection locations and/or connection elements may be pre-existing parts for cooperation with an outer shell, but repurposed for cooperation with the adapter. The connection locations may be abutment locations and/or abutment elements. The connection locations may be pre-existing abutment locations and/or abutment elements for cooperation with abutment elements and/or abutment locations on an outer shell repurposed for cooperation with abutment elements and/or abutment locations of the adapter.

A cemented type liner may be devoid of connection locations and/or connection elements. A cemented type liner may be devoid of connection locations and/or connection elements which are discrete parts extending from and/or recess relative to the remainder of the liner. The method may include selecting the outer shell from amongst a plurality of outer shells, at least one of the outer shells having one or more different outer shell characteristics than another one of the outer shells.

The method may include selecting the adapter from amongst a plurality of adapters, at least one of the adapters having one or more different adapter characteristics than another one of the adapters.

The method of assembly may be provided as part of a method of surgery. The method of surgery may include preparing a site for the outer shell, for instance on the acetabulum. The method of surgery may include mounting the outer shell on the patient. The method of surgery may include attaching the adaptor and the liner to the outer shell. The method may include a cement free attachment of the adaptor and the liner to the outer shell.

The method of assembly may include inserting the liner into the adaptor. The method may include the insertion being through relative axial movement of the liner and adaptor.

The method may include inserting the adaptor into the outer shell.

The method may include the liner being in the adaptor when the adaptor is inserted into the outer shell. The method may include the insertion being through relative axial movement of the liner and/or adaptor to the outer shell. The method may include the insertion being through relative rotational movement of the liner and/or adaptor to the outer shell.

The method of assembly may use no cement. The method of assembly may use no cement to connect the outer shell to the adapter. The method of assembly may use no cement to connect the adapter to the liner. The method of assembly may use no cement to connect the outer shell to the adapter and to the liner. The method of assembly may use no cement to connect the liner to the adapter and/or outer shell. The method of assembly may use no cement in contact the distal side of the outer shell and/or proximal side of the adapter and/or distal side of the adapter and/or proximal side of the liner and/or distal side of the liner. The method may include cement being provided in contact with the proximal side of the outer shell.

The method may further comprise the use of one or more further outer shells, at least one of the outer shells having one or more different shell characteristics than another one of the outer shells. The method may further comprise the use of one or more different characteristics between outer shells in that one or more outer shells is a cemented outer shell and/or one or more outer shells is a non-cemented outer shell.

A non-cemented outer shell may be provided with one or more connection locations and/or connection elements. The connection locations and/or connection elements may be discrete parts extending from and/or recess relative to the remainder of the outer shell. The connection locations and/or connection elements may be pre-existing parts for cooperation with a liner, but repurposed for cooperation with the adapter. The connection locations may be attachment locations and/or attachment elements. The connection locations may be pre-existing attachment locations and/or attachment elements for cooperation with attachment elements and/or attachment locations on a liner repurposed for cooperation with attachment elements and/or attachment locations of the adapter.

A cemented outer shell may be devoid of connection locations and/or connection elements. A cemented outer shell may be devoid of connection locations and/or connection elements which are discrete parts extending from and/or recess relative to the remainder of the outer shell.

A cemented outer shell may be an outer shell adapted for fixing to the patient by cement. A non-cemented outer shell may be an outer shell adapted for fixing to the patent other than by cement, for instance by one or more bone fixings.

A non-cemented shell may be provided with one or more mounting locations, such as fixation mounting locations. The one or more fixation mounting locations may be apertures and/or slots extending through the non-cemented shell. The one or more fixation mounting locations may receive a fixation, in use, such as a screw and/or nail and/or pin. The fixation may be a bone engaging fixation. The one or more fixation mounting locations may be provided with an expanded profile section, for instance to receive a head portion of the fixation, in use.

A cemented shell may be devoid of mounting locations, potentially fixation mounting locations, such as apertures or slots extending through the cemented shell.

The cemented shell may be provided with one or more cement contacting surfaces. The cemented shell may physically and/or chemically interact with cement on contact.

The third aspect of the disclosure may include any of the features, options or possibilities set out elsewhere in this document, including in the other aspects of the disclosure.

According to a fourth aspect of the disclosure there is provided a method of implanting a component of a prosthesis, the method comprising:
obtaining an adaptor, a liner and an outer shell;
inserting the liner into the adapter;
inserting the adapter into the outer shell;
implanting the adaptor, the liner and the outer shell at a surgical site;
   wherein the component comprises:
an outer shell;
a liner provided at least partially within the outer shell and adapted to provide a contact surface for a further component of the prosthesis; and
an adaptor provided at least partially within the outer shell and with the liner provided at least partially within the adaptor.

The method may further provide the acetabular component in an assembled state and in the assembled state relative articulation between the adaptor and the outer shell is prevented. The method may further provide the acetabular component in an assembled state and in the assembled state relative articulation between the adapter and the liner is prevented. The method may further provide the acetabular component in an assembled state and in the assembled state relative articulation between the adaptor and the outer shell is prevented and in the assembled state relative articulation between the adapter and the liner is prevented.

The method may include the component being an acetabular component. The component may be a component of an orthopaedic joint prosthesis. The outer shell may be for mounting on the acetabulum. The further component may be a femoral component, for instance of an orthopaedic joint prosthesis.

The method may include selecting the liner from amongst a plurality of liners, at least one of the liners having one or more different characteristics than another one of the liners. The one or more different characteristics between liners may be that one or more liners is a cemented liner and that one or more liners is a non-cemented liner, particularly non-modified cemented-type liners and non-modified non-cemented type liners.

A non-cemented type liner may be provided with one or more connection locations and/or connection elements. The connection locations and/or connection elements may be discrete parts extending from and/or recess relative to the remainder of the liner. The connection locations and/or connection elements may be pre-existing parts for cooperation with an outer shell, but repurposed for cooperation with the adapter. The connection locations may be abutment locations and/or abutment elements. The connection locations may be pre-existing abutment locations and/or abutment elements for cooperation with abutment elements and/or abutment locations on an outer shell repurposed for cooperation with abutment elements and/or abutment locations of the adapter.

A cemented type liner may be devoid of connection locations and/or connection elements. A cemented type liner may be devoid of connection locations and/or connection elements which are discrete parts extending from and/or recess relative to the remainder of the liner. The method may include selecting the outer shell from amongst a plurality of outer shells, at least one of the outer shells having one or more different outer shell characteristics than another one of the outer shells.

The method may include selecting the adapter from amongst a plurality of adapters, at least one of the adapters having one or more different adapter characteristics than another one of the adapters.

The method of surgery may include preparing a site for the outer shell, for instance on the acetabulum. The method of surgery may include mounting the outer shell on the patient. The method of surgery may include attaching the adaptor and the liner to the outer shell. The method may include a cement free attachment of the adaptor and the liner to the outer shell.

The method may include inserting the liner into the adaptor. The insertion may be through relative axial movement of the liner and adaptor.

The method may include inserting the adaptor into the outer shell.

The method may include that the liner may be in the adaptor when the adaptor is inserted into the outer shell. The method may include, the insertion being through relative axial movement of the liner and/or adaptor to the outer shell. The method may include the insertion being through relative rotational movement of the liner and/or adaptor to the outer shell.

The method may use no cement in the implanting. The method may use no cement to connect the outer shell to the adapter. The method may use no cement to connect the adapter to the liner. The method may use no cement to connect the outer shell to the adapter and to the liner. The method may use no cement to connect the liner to the adapter and/or outer shell. The method may use no cement in contact the distal side of the outer shell and/or proximal side of the adapter and/or distal side of the adapter and/or proximal side of the liner and/or distal side of the liner. The method may include cement being provided in contact with the proximal side of the outer shell.

The method may further comprise the use of one or more further outer shells, at least one of the outer shells having one or more different shell characteristics than another one of the outer shells. The method may further comprise the use of one or more different characteristics between outer shells in that one or more outer shells is a cemented outer shell and/or one or more outer shells is a non-cemented outer shell.

A non-cemented outer shell may be provided with one or more connection locations and/or connection elements. The connection locations and/or connection elements may be discrete parts extending from and/or recess relative to the remainder of the outer shell. The connection locations and/or connection elements may be pre-existing parts for cooperation with a liner, but repurposed for cooperation with the adapter. The connection locations may be attachment locations and/or attachment elements. The connection locations may be pre-existing attachment locations and/or attachment elements for cooperation with attachment elements and/or attachment locations on a liner repurposed for cooperation with attachment elements and/or attachment locations of the adapter.

A cemented outer shell may be devoid of connection locations and/or connection elements. A cemented outer shell may be devoid of connection locations and/or connection elements which are discrete parts extending from and/or recess relative to the remainder of the outer shell.

A cemented outer shell may be an outer shell adapted for fixing to the patient by cement. A non-cemented outer shell may be an outer shell adapted for fixing to the patent other than by cement, for instance by one or more bone fixings.

A non-cemented shell may be provided with one or more mounting locations, such as fixation mounting locations. The one or more fixation mounting locations may be apertures and/or slots extending through the non-cemented shell. The one or more fixation mounting locations may receive a fixation, in use, such as a screw and/or nail and/or pin. The fixation may be a bone engaging fixation. The one or more fixation mounting locations may be provided with an expanded profile section, for instance to receive a head portion of the fixation, in use.

A cemented shell may be devoid of mounting locations, potentially fixation mounting locations, such as apertures or slots extending through the cemented shell.

The cemented shell may be provided with one or more cement contacting surfaces. The cemented shell may physically and/or chemically interact with cement on contact.

The fourth aspect of the disclosure may include any of the features, options or possibilities set out elsewhere in this document, including in the other aspects of the disclosure.

### BRIEF DESCRIPTION OF THE DRAWINGS

FIG. 1 is a schematic illustration of a kit according to a general embodiment of the disclosure showing a shell, a series of different liners and an adapter;

FIG. 2a is a perspective side view of an adapter and a shell according to a first detailed embodiment of the disclosure;

FIG. 2b is a side sectional view, showing the adapter 100 and a selected liner 5 positioned within it in a part assembled form of the implant 1;

FIG. 2c is a side sectional view, showing the adapter 100 engaged with the shell 3, with the adapter 100 sandwiched between the shell 3 and the liner 5;

FIG. 3 is a side detail view of the locking element of a variant embodiment of an adapter;

FIG. 4 is a schematic illustration of a kit according to a second general embodiment of the disclosure showing a shell, a series of different liners and an adapter;

FIG. 5a is a perspective view of a shell and adapter according to a second detailed embodiment of the disclosure;

FIG. 5b is a cross-sectional side view of the adapter only of FIG. 5a;

FIG. 5c is a plan view of the adapter only of FIG. 5b;

FIG. 5d is a cross-sectional side view of the shell, the adapter and a selected liner of FIG. 5a in an assembled state;

FIG. 5e is a cross-sectional side view of the shell and the adapter of FIG. 5a, with a different selected liner;

FIG. 5f is a cross-sectional side view of the shell and the adapter of FIG. 5a, with a further different selected liner;

FIG. 6a is a plan view of an adapter according to a first variant of the second detailed embodiment;

FIG. 6b is a detailed side view of the arms in a second variant of the second detailed embodiment;

FIG. 7 is a detailed plan view of the arms in a third variant of the second detailed embodiment;

FIGS. 8a and 8b are two positions for a detailed part of the periphery of an adaptor according to a fourth variant of the second detailed embodiment; and

FIG. 9 is a cross-sectional side view of an adapter according to a third detailed embodiment connected to a base of a shell.

### DETAILED DESCRIPTION OF THE DRAWINGS

In orthopaedic uses, a variety of different acetabular cup replacement approaches can be taken. Only a few of these are suitable to be used in complex acetabular defect surgery where more traditional shells with fixings that engage the bone cannot be used successfully. Fully porous shells with cemented in type liners provide one such approach. However, the fully porous shell is designed to be complimentary to a given size and type of liner that is cemented to it. Thus, the liner material, liner size and position and potentially other characteristics are constrained for any given porous shell and so such an approach is unable to offer as great a range of sizes and positions for the implant as modular systems. For any given size of modular system shell a much wider variety of liners made from different materials, with different size, different cup positions and the like are already made available.

Modular liners can achieve the variation, but they are designed for traditional shells that use fixings to mount them and which do not employ cement between the shell and the liner.

Presently there is no provision for the modular system liners to be securely attached to fully porous shells, presently only intended for use with cemented type liners. Thus, there are limits on the user's freedom to select different bearing styles and/or materials, for instance to restore hip centre.

Porous shells are beneficial for bone ingrowth and fully porous shells seek to mimic the cancellous bone throughout. Other porous shells just have a porous coating on the outer surface, proximal surface, and so do not mimic all of the way through the shell.

As well as being useful in hip implant systems, a similar improvement would also be beneficial in allowing the attachment of modular components in cage type implants.

A common feature to cemented shells and their liners is that the cement provides the attachment there between and so no other form of attachment location or attachment element are pre-existing on the shell or liners.

The disclosure provides an adapter for use between the porous shell and either type of the liner in an implant, to allow a wider range of liners to be used with a given shell. The disclosure is suitable for use in a porous shell and liner-based implant, but allows a given porous shell to successfully work with a range of different liners and potentially accept modular liners as well as previously cemented type liners. These forms can be obtained for non-modified cement-type and non-modified non-cement type liners, both of which would now be used in non-cement incorporating forms. A greater range of treatment options can result.

In other embodiments, the approach is used with cage and other types of implant to offer similar flexibility.

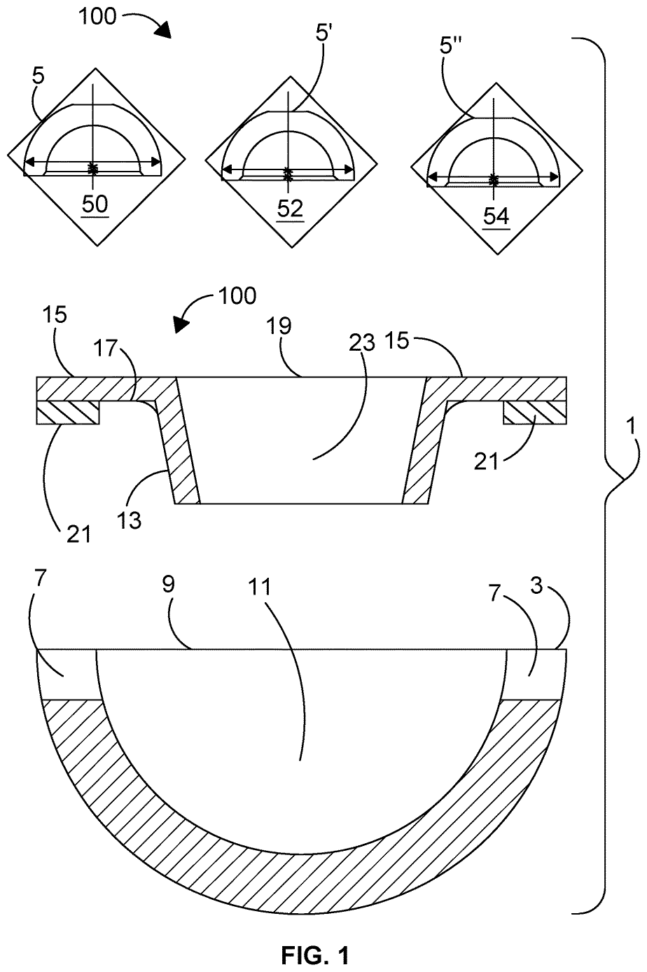

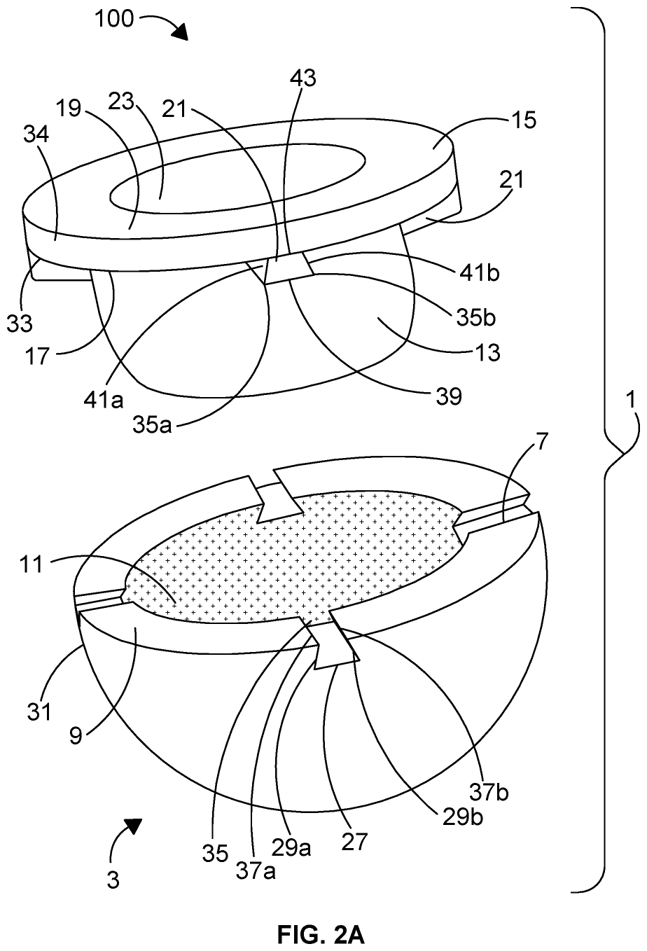

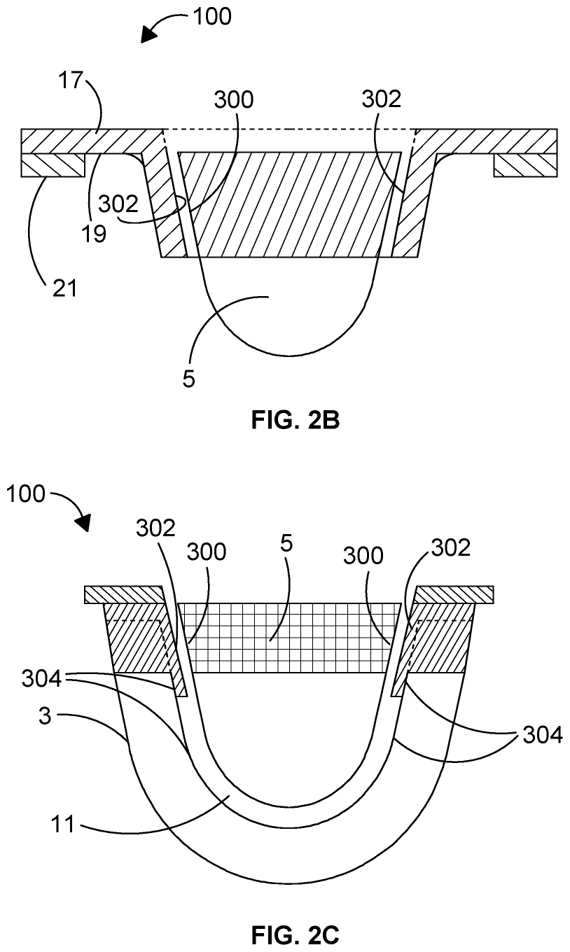

In FIG. 1, a kit according to the disclosure, particularly of the type using a shell 3 and an adapter 100 shown in more detail in FIG. 2, is illustrated. The kit is used to provide an overall implant 1 formed of a shell 3 and an adapter 100, in combination with a single liner 5 selected from a group of different diameter liners 5, 5′ and 5″. Three liners: 5 with diameter 50 mm, 5′ with diameter 52 mm and 5″ with diameter 54 mm are shown, but a larger number or just two liners 5, 5′ could feature in the kit.

The porous shell 3 is provided with attachment locations 7 on a distal surface 9 of the shell 3. The distal surface 9 surrounds a dished recess 11. The dished recess 11 is configured to accept a body 13 of an adapter 100. The adapter 100 has a perimeter flange 15 with a proximal surface 17 and a distal surface 19. In the assembled state, at least a part of the proximal surface 17 of the adapter 100 abuts at least a part of the distal surface 9 of the shell 3. The proximal surface 17 of the adapter 100 is provided with a series of attachment elements 21.

The adapter 100 is provided with a recess 23 in its distal surface 19. The recess 23 has a defined size [such as diameter and/or depth], a defined position within the adapter 100 [and hence in the shell 3 and the overall implant 1], and a defined cup internal profile [for instance hemispherical or tapering truncated cone as shown in FIG. 1]. The recess or cup 23 receives the selected liner 5.

The kit includes the liner 5 and a series of other liners 5′, 5″, etc that differ from the liner 5 in terms of one of more characteristics. The characteristics are generally distal characteristics, for instance a different defined size [such as a larger diameter and/or deeper depth to accommodate a larger ball on a different implant], and/or a different defined position within the liner 5 [such as a non-central position], and/or a different defined cup internal profile [for instance not a purely hemispherical profile]. Typically, the existing range of modular type liners [non-cemented type], each offering variation in such characteristics, is greater than the range of cemented type liners constructed. The characteristics may alternatively or additionally be generally proximal characteristics and/or characteristics relating to the interaction of the liner 5, 5′, 5″etc with the adapter 100. Typically, the existing non-cemented type liners may include a connection location and/or connection element intended to cooperate with the outer shell of the original designs, but now potentially put to alternative use to cooperate with the adapter. In contrast, the existing liners intended for cemented use are typically devoid of such discrete elements and rely upon the cement to retain them in the outer shell.

The adapter disclosed now provides an abutment style engagement in place of the cement for the cement type liners [without the need to modify those liners] and also uses the same abutment style engagement to retain the non-cemented type liners [again without the need for modification] and even where those feature connection locations and/or connection elements.

As a result of the kit, a given shell 3 can optionally be combined with any of the liners 5, 5′, 5″, etc. successfully to provide variation in the liner 5, 5′, 5″etc and hence in the overall implant 1.

Whilst a single adapter 100 may be used and is shown in FIG. 1, the kit could include a number of adapters 100, 100′, 100″ etc. where the number of adapters is materially fewer than the number of liners 5 in the same kit.

If more than one adapter 100 is provided, then the adapters may have one or more non-common characteristics. The characteristics are generally distal characteristics, for instance a different defined size [such as a larger diameter and/or deeper depth to accommodate a larger ball on a different implant], and/or a different defined position within the adapter 100 [such as a non-central position], and/or a different defined cup internal profile [for instance not a purely hemispherical profile].

If more than one adapter 100 is provided, then the adapters may have one or more common characteristics. The common characteristics may include the perimeter flange 15, such as the proximal surface 17 thereof, having a common profile and/or size and/or extent. The common characteristics may include the nature and/or size and/or shape and/or profile of the body 13 of the adapter 100, for instance such that it is a secure fit within the shell 3. The common characteristics may include the nature and/or size and/or shape and/or profile and/or position of the attachment locations 7 and attachment elements 21.

As illustrated in FIG. 1, only a single shell 3 is shown for simplicity, but the disclosure for the kit and generally includes a series of different shells 3 which vary from one another in terms of one or more shell characteristics. These may be shell distal characteristics, such as the nature and/or size and/or shape and/or profile of the shell distal side or part thereof, for instance the dished recess 11, and/or they may be shell proximal characteristics, such the nature and/or size and/or shape and/or profile of the shell proximal side or part thereof, for instance one or more characteristics of the porous coating on the shell 3 and/or external shape of the shell 3. As a result, a wide variety of different shells 3, potentially different adapters 100 and different liners 5, 5′, 5″, 5′″, 5″″, etc can be combined, whilst retaining the benefits of a porous shell 3 type design.

Referring to FIG. 2a, a shell 3 and an adapter 100 suitable for use in such a general kit is illustrated. In FIG. 2b, that adapter 100 and a selected liner 5 are shown in a part-assembled form of the implant 1. In FIG. 2c, the adapter 100 is engaged with the shell 3, with the adapter 100 sandwiched between the shell 3 and the liner 5, to give the assembled form of the implant 1.

This first detailed embodiment provides a porous shell 3 with an external, proximal surface 23 provided with a porous coating, as known in the art. Four equally spaced and matching profile attachment locations 7 are provided recessed in the distal surface 9 of the shell 3. The distal surface 9 surrounds a dished recess 11. The dished recess 11 is configured to accept the body 13 of the adapter 100 [shown detached from but aligned with the shell 3]. The shell 3 and the attachment locations 7 can readily be formed by 3-D printing processes.

Attachment locations within the dished recess 11 could also be employed, alternatively or additionally. For instance, an attachment location could be provided in the base of the shell 3, for instance using an aperture, from one or more apertures provided in the shell 3 for potential use with a fixing; FIG. 9 illustrates such a use.

The attachment locations 7 have a mouth 25 and a base 27. The mouth 25 is connected to the base 27 by two inclined surfaces 29a, 29b. The inclined surfaces 29a, 29b, flare away from the mouth 25 and away from each other such that the base cross-section is greater than the mouth cross-section.

The adapter 100 has a perimeter flange 15 with a proximal surface 17 and a distal surface 19. In the assembled state, the proximal surface 17 of the adapter 100 is aligned with and abuts with the distal surface 9 of the shell 3. A smooth continuation is provided between the adjacent portion 31 of the external, proximal, surface 23 of the shell 3 and the further adjacent portion 33 of the external surface 34 of the adapter 100.

The proximal surface 17 of the adapter 100 is provided with the series of attachment elements 21. During transition from the disassembled state FIG. 2 to the assembled state, the attachment elements 21 are aligned with respective attachment locations 7 and compressive force is applied, such that the edge parts 35a, 35b of each of the attachment elements 21 deform due to abutment with the edge surfaces 37a, 37b of the distal surface 9 of the shell 3. Further compression causes further deformation until the attachment elements 21 snap into position in the attachment locations 7. The attachment elements 21 are each profiled with a proximal surface 39 and inclined side surfaces 41a, 41b leading to a neck location 43. These surfaces and locations are complimentary to those of the attachment locations 7 of the shell 3, at least with respect to the abutting surfaces, and hence provide a firm fixing between porous shell 3 and adapter 100.

Referring to FIG. 2b, a liner 5 has been selected from amongst a set of different liners 5, 5′, 5″ etc, for use in forming the implant 1. The liner 5 is inserted in the recess 23 in the adapter 100. The clearances in FIG. 2c are exaggerated, with the outer surface 300 of the liner 5 approaching or abutting the inner surface 302 of the recess 23. Little or no compression of the liner 5 by the adapter 100 may occur in this configuration. The adapter 100 has the distal surface 17, proximal surface 19 and attachment elements 21.

In FIG. 2c, the partially assembled implant 1 has been changed into the complete assembled implant 1 by inserting the adapter 100, carrying the liner 5, into the shell 3. The type of engagement between the adapter 100 and the shell 3 is describe above in detail with reference to FIG. 2a. The surface 304 defining the dished recess 11 in the shell 3 abuts with the outer surface 306 on the adapter 100 at abutment locations. The inner surface 302 of the adapter 100 abuts with the outer surface 300 of the liner 5 at abutments locations. The abutment may be such that the liner 5 compresses the adapter 100 and/or the adapter 100 compresses the liner slightly. Compression of the shell 3 and/or the outer surface of the adapter 100 may also be caused. The abutment, potentially with compression, provides firm positioning of the liner 5 in the adapter 100. The adapter 100 is firmly anchored to the shell 3 also.

Thus, as immediately described, the connection of the liner 5 with the adapter 100 is a mechanical one and is achieved through abutment. This works for the non-cemented type liners, even though no engagement between connection locations and/or connections elements provided by the liner occurs with the adapter; such connection may be used in liner to outer shell direct engagements previously. This mechanical interaction is significant in the context of a liner previously designed for cemented use, as the cement used to provide the fixing in typical porous shell and liner is absent. Despite this, the cemented-type liner is firmly held in position. As described below the adapter is sufficiently adaptable in combination with the outer shell to successfully retain even different outside dimensioned liners and without the use of connection elements and/or connection locations.

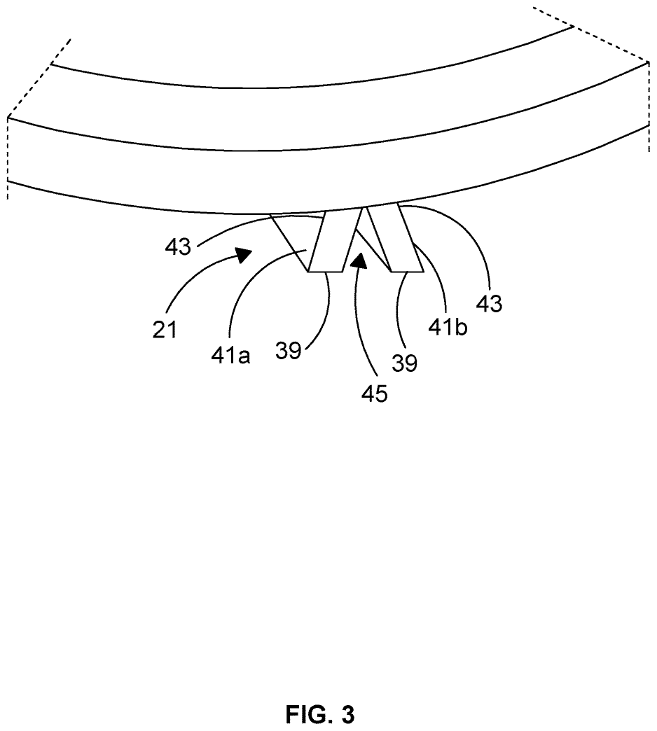

An alternative variant for the attachment elements 21 is shown in FIG. 3. In this case, the attachment element 21 is provided with a central wedge-shaped recess 45 absent from the type of attachment elements 21 shown in FIG. 2. This provides for greater flexibility in the attachment elements 21 during the transition from disassembled state to assembled state. The attachment element 21, as would the others not shown, is provided with the proximal surface 39 [split into two parts] and the inclined side surfaces 41a, 41b that lead to the neck location 43. These surfaces and locations are complimentary to those of the attachment locations 7 of the shell 3, at least with respect to the abutting surfaces, and hence provide a firm fixing between porous shell 3 and adapter 100. The attachment locations 7 of the shell 3 in this variant can have the same profile as shown in FIG. 2, but may alternatively have some variation, particularly with respect to the profile of the base 27, such as a protrusion [not shown] into the central wedge-shaped recess 45.

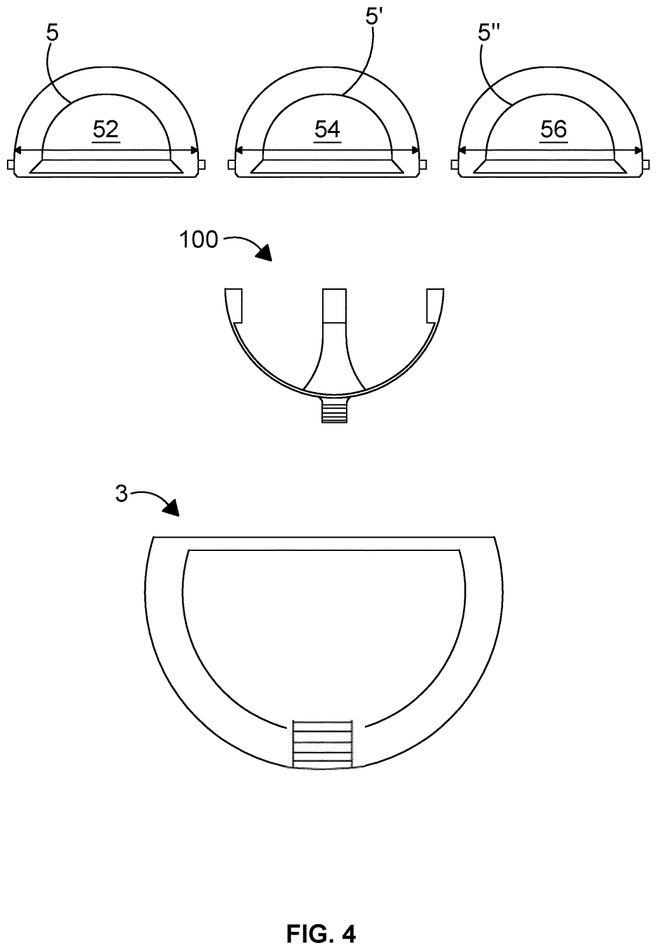

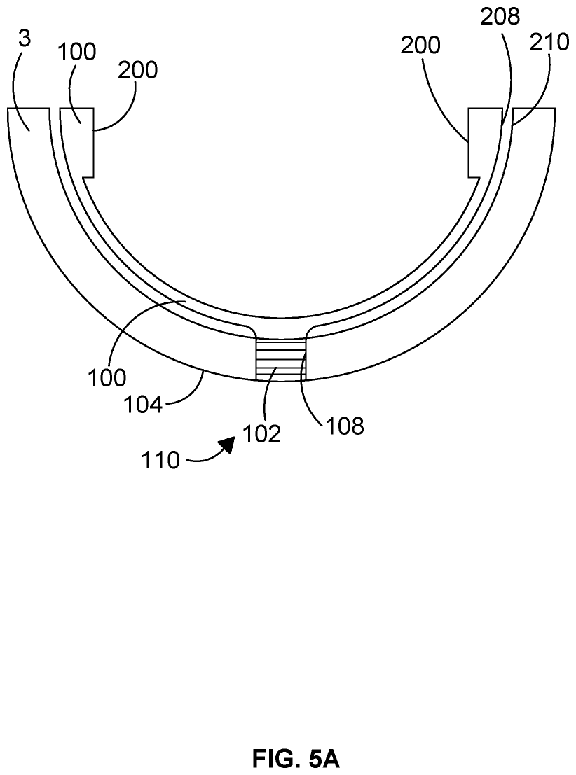

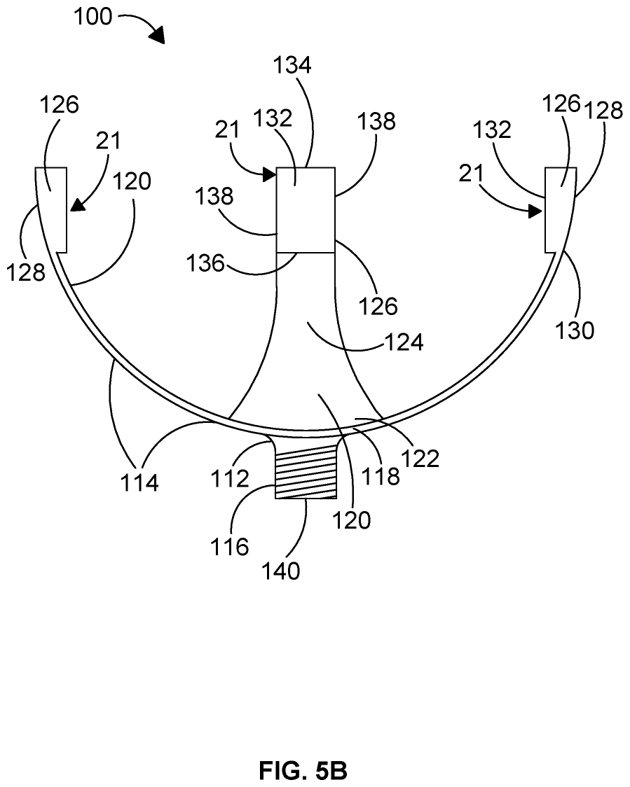

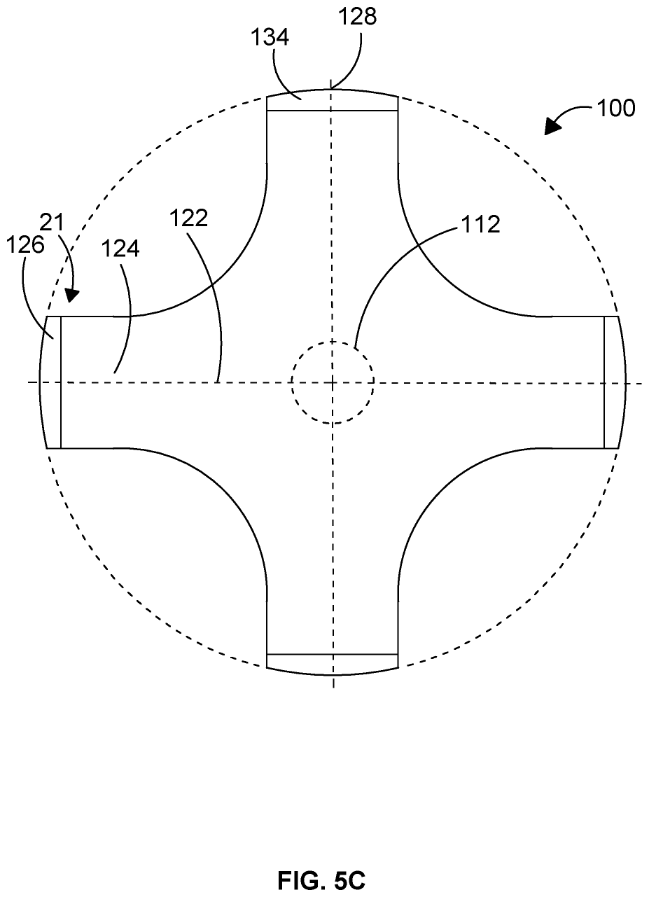

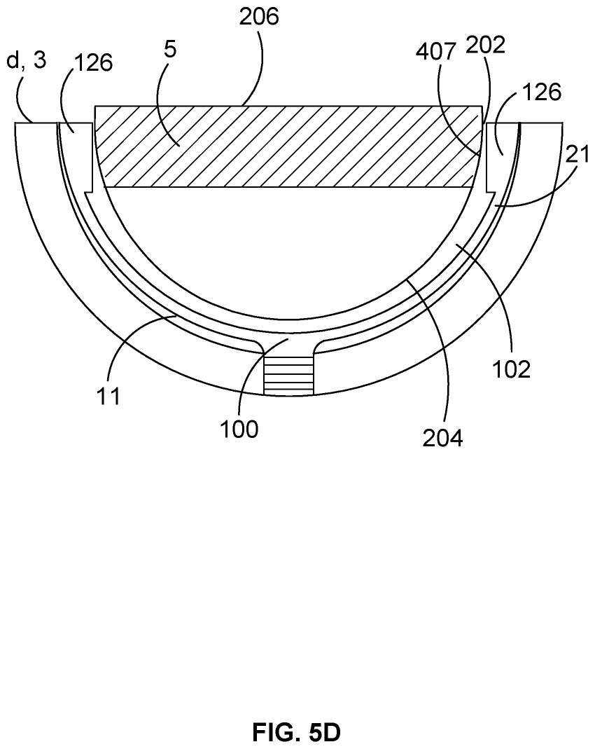

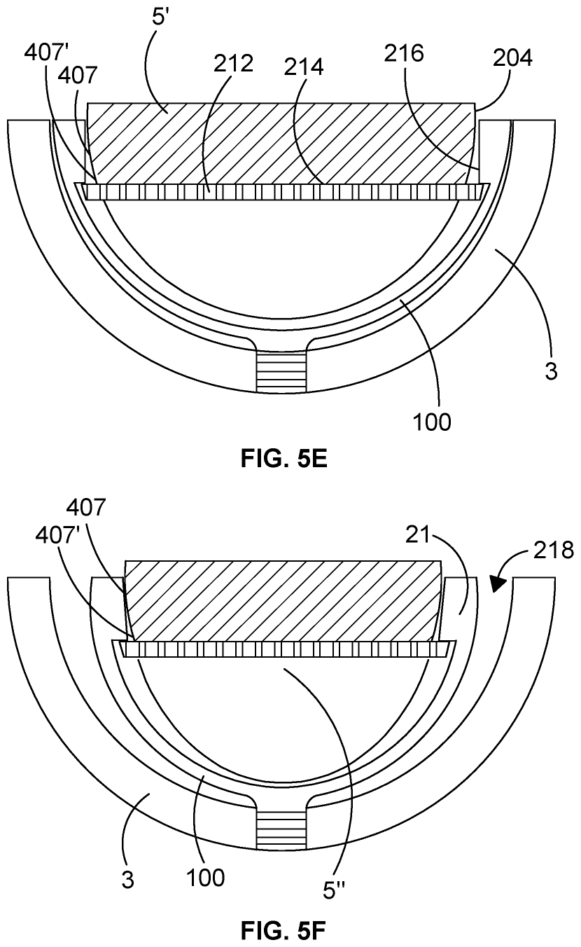

FIG. 4 is a schematic illustration of a kit according to a second general embodiment of the disclosure. Once again, it features a shell 3 and a liner 5, together with an adapter 100. The shell 3 and liner 5 may be of the type shown in more detail in FIG. 5d, FIG. 5e and FIG. 5f. The adapter 100 may be of the type shown in more detail in FIGS. 5a, 5b, 5c, 5d, 5e and 5f. As well as the liner 5 with a first external diameter of 52 mm, the kit includes at least one other liner. In this case liner 5′ with an external diameter of 54 mm and liner 5″ with an external diameter of 56 mm are provided. The liners 5, 5′, 5″ are shown rotated through 180° compared with their orientation when inserted into the adapter 100 and shell 1.

The adapter 100, in the assembled state shown in FIG. 5d, and during assembly in FIG. 5a, is provided between the shell 3 and the liner 5. The adapter 100 is received in the dished recess 11 in the shell 3. The adaptor 100 has a central space 102 and the liner 5 is received in that central space 102. In general terms, the adaptor 100 engages with the shell 3 through attachment elements/locations at the base, may have abutment locations/elements at its distal ends and engages with the liner 3, through abutment locations/elements of the liner, so as to firmly fix the three parts together and provide a firmly assembled implant 1.

A single adapter 100 provides the ability to successfully mount in a given shell 3, a larger range of liners 5, than would be possible with a liner that is mounted directly in a shell. Thus, in the illustrated embodiment, the shell 3, through the use of a single adaptor 100 is able to successfully accommodate a choice from a number of liners 5, 5′, 5″ and 5″, for instance. The different liners 5, 5′, 5″ can be accommodated by a single adapter 100 as the end sections 126 can assume different spacings from each other within the void 75; shown in FIGS. 5d, 5e and 5f.

If, as is envisaged, the kit includes a second different adaptor, then that too is able to allow a shell 3 to successfully receive further different liners for instance. The addition of still further different adaptors to the kit would allow an even greater range of liners to be used. It would be possible to provide the adaptors 100 such that at liner ranges that can be accommodated overlap in their ranges. Thus, a fourth and subsequent sized liners might be accommodated in adaptor 100 and a larger adaptor, whereas liner 5, 5′ and 5″ would not be accommodated in that larger adaptor, and so on.

As with the FIG. 1 embodiment above, whilst only a single shell 3 is shown in FIG. 4 for simplicity, the disclosure for the kit and generally includes a series of different shells 3 which vary from one another in terms of one or more shell characteristics. Thus, a greater range of shells and of liners that can be fitted within them is provided by the use of the adaptor and potentially different adaptors.

In FIG. 4, a porous shell 3 and a liner 5 are shown of the type that the adaptor 100 shown in FIGS. 5a, 5b and 5c can accommodate.

Compared with a conventional porous shell, the shell 3 has an aperture 102, provided in the base 104 of the dished recess 11. The aperture 102 may have been added or modified and provides a threaded section 108 as part of a shell connection location 110; attachment locations/elements.

Compared with the operation of a conventional liner cemented in a shell, as seen best in FIGS. 5d, 5e and 5f, the liner 5 has a series of abutment locations 407, provided on the external surface 35 of the liner 5. These abutment locations 407 cooperate with abutment locations or elements 21 provided with end sections 126, which in this embodiment are provided by the adaptor 100, FIG. 5b.

If the cemented type liner is modified compared with existing forms thereof, then the abutment locations 407 could be in the form of an attachment location provided by a peripheral open slot provided with an inner face, side faces and base face, which base face faces proximally. The attachment locations 407 have the same profile in each instance.

Such abutment locations 407 could provide increased retention of the liner 5 in the adapter 100 and hence shell 3.

Otherwise, and as shown in FIG. 5d, the shell 3 and the liner 5 can be unmodified relative to existing porous shell and cemented liner designs, with the internal faces 200 of the end sections 126 on the attachment elements 21 of the adapter 100 merely abutting the tapered surface 202 on the external side 204 of the liner 5 and near the distal side 206 of the liner 5. The external faces 208 of the end sections 126 abut the internal perimeter section 210 of the shell 100. A firm engagement for the shell 3, adapter 100 and liner 5 are thus provided.

Alternative abutment locations 407 are provided by the liner 5′ in FIG. 5e and the liner 5″ in FIG. 5f.

In the FIG. 5e form, the liner 5′ has a similar abutment location 407 to that of FIG. 5d where the liner shell 3, adapter 100 and liner 5′ abut, but also has further abutment location 407′ on the liner 5′. The further abutment location 407′ is provided by an annular ring 212 which extends around and projects from the external surface 204 of the liner 5′. The distal surface 214 of the annular ring 212 provides an abutment and restraining surface in cooperation with the inner, proximal surface 216 of the attachment elements 21 of the adapter 100.

In the FIG. 5f form, the liner 5″ has the attachment approach of FIG. 5e, but the liner 5″ is smaller than the liner 5′ in FIG. 5e and so the adapter 100 does not have its attachment elements 21 in abutment with the shell 3 and a gap 218 exists.

In general, during assembly, the liner 5 is introduced into the adaptor 100 first, to give the assembled liner-adaptor state. The combined liner 5 and adaptor 100 can then be attached to the shell 3 to provide the assembled state for the implant 1.

In FIGS. 5b and 5c the adaptor 100 is illustrated in detail. To provide the firm engagement between the adaptor 100 and the shell 3, a base stem 112 is provided centrally on the proximal surface 114 of the adaptor 100. The base stem 112 has a threaded part 116 which cooperates with the threaded section 108 provided in the aperture 102 of the shell connection location 110 of the shell 3. The base stem 112 extends from an adaptor base 118. This provides the attachment locations/elements.

The adaptor base 118 has four arm elements 120 that extend outwardly and distally therefrom. Each of the arm elements 120 has the same shape and configuration. The arm elements 120 are provided in two opposing pairs. The arm elements 120 have a base section 122 which links them to the adaptor base 118, an intermediate section 124 which is narrower, considered as a circumferential extent, and an end section 126. The intermediate section 124 provides the main ability for the arm elements 120 to flex radially outwardly and inwardly as needed.

Each of the end sections 126 is provide with an abutment element 21. The abutment elements 21 are shown with an equivalent profile in each case. In this embodiment, the abutment elements 21 have an external surface part 128 which is a smooth continuation of the external arm surface 130. The external arm surface 130 and the external surface part 128 may have a profile complimentary to the part of the shell 3 that they abut in the assembled state. The abutment elements 21 also have an internal surface 132, a distal surface 134, a proximal surface 136 and side surfaces 138. The internal surface 132 provides a tapered engagement area that abuts the liner 5 in use.

In the assembled state, and where the liner 5 is in the modified form including peripheral open slots that contribute to the abutment locations, the proximal surface 136 of the abutment elements 21 abuts with the base face 206 of the liner 5. The internal surface 132 of the abutment element 21 abuts the inner face 202 of the liner 5 and the side surfaces 138 of the abutment element 21 abut the side faces 204 of the liner 5. The abutment elements 21 generally have a profile complimentary to the peripheral open slots 200 provided by the liner 5.

As the liner 5 is introduced into the adaptor 100, proximally, the external surface 35 of the liner 5 will come into contact with a location on the arm elements 120 and further movement of the liner 5 into the adaptor 100 will cause the arm elements 120 to flex outwardly. Eventually, the abutment elements 21 on the arm elements 120 will align with the abutment locations 407 of the liner. This may be through axial movement of the adaptor 100 and the liner 5 with the abutment elements 21 and the abutment locations 407 or aligned, or through rotation of the adaptor 100 relative to the liner 5 to being the abutment elements 21 and the abutment locations 407 into alignment, or a combination of both motions. Once the abutment elements 21 align with the abutment locations 407, then the abutment locations 21 are able to enter the abutment locations 407 and the arms are able to return radially inward and the flexing of the arm elements 120 is eased.

In this assembled liner-adaptor state, the liner 5 and adaptor 100 combination can be introduced to the shell 3; to give the FIG. 5d fully assembled form. The liner 5 and adaptor 100 combination is inserted, proximally, into the dished recess 11 of the shell 3 and manoeuvred to provide the proximal end 140 of the base stem 112, provided centrally on the proximal surface 114 of the adaptor 100, in the aperture 102 of the shell connection location 110 of the shell 3. Relative rotation of the liner 5 and adaptor 100 combination relative to the shell 3 results in the threaded part 116 of the base stem 112 engaging with and advancing along the threaded section 108 provided in the aperture 102 in the base 104 of the dished recess 11 of the shell 3. Continued rotation brings the external surfaces of the adaptor 100 into closer proximity with the internal surfaces of the shell 3. As the proximal surface 114 of the adaptor 100 abuts the distal surface of the shell 3, the profiles of the shell 3 and the adaptor 100 are such that the distal section 146 of the dished recess 11 abuts the external arm surface 130 and the external surface part 128 of the arm elements 120 of the adaptor 100. This applies a compressive force on the abutment elements 21 of the adaptor 100 into the abutment locations 407 of the liner 5; a locking ring style action is provided. A firm engagement between shell 3 and adaptor 100 is thus provided and this also serves to increase the firm engagement between the adaptor 100 and the liner 5; the assembled implant state is reached.

The shell 3 is able to accommodate different liners 5 in this disclosure as the adaptor 100 is able to flex radially outwardly and radially inwardly over a material range so as to accommodate different dimensions of liner 5. In effect, the adaptor 100 is mainly within the void 75 filled by cement in the prior art where just a shell 3 and liner 5 were used. The void 75 is free of cement when the adapter 100 is used to provide the shell 3 to liner 5 connection. At the same time, the adaptor 100 is still able to attach firmly to the shell 3 and firmly clamp to the liner. Referring to FIG. 5e, a first sized liner 5′ is used which is the maximum size that can be accommodated by this adapter 100. In FIG. 5f, a smaller liner 5″ is used and so the adapter 100 has its attachment elements 21 less widely deformed and a gap 218 exists between the adapter 100 and the shell 3.

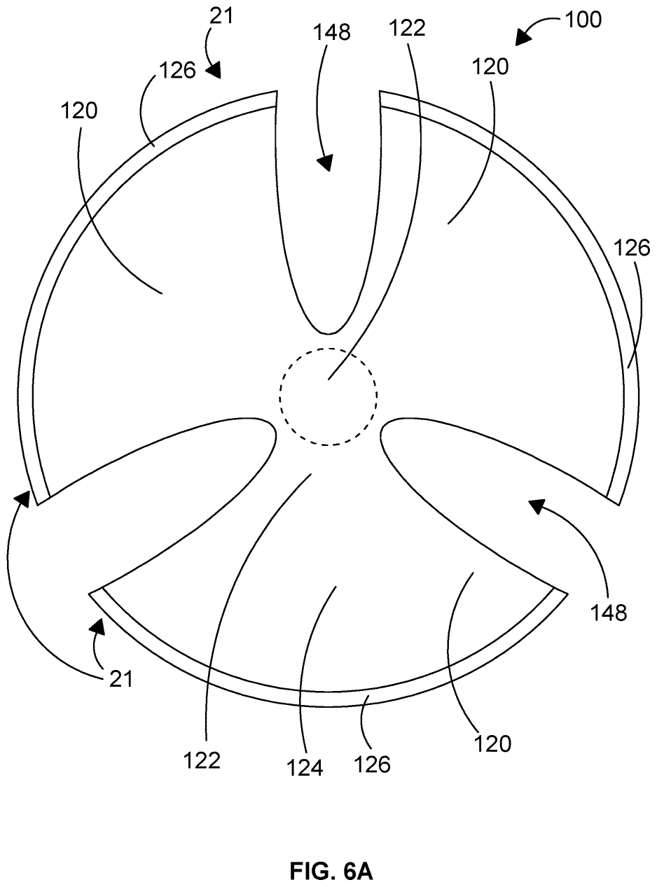

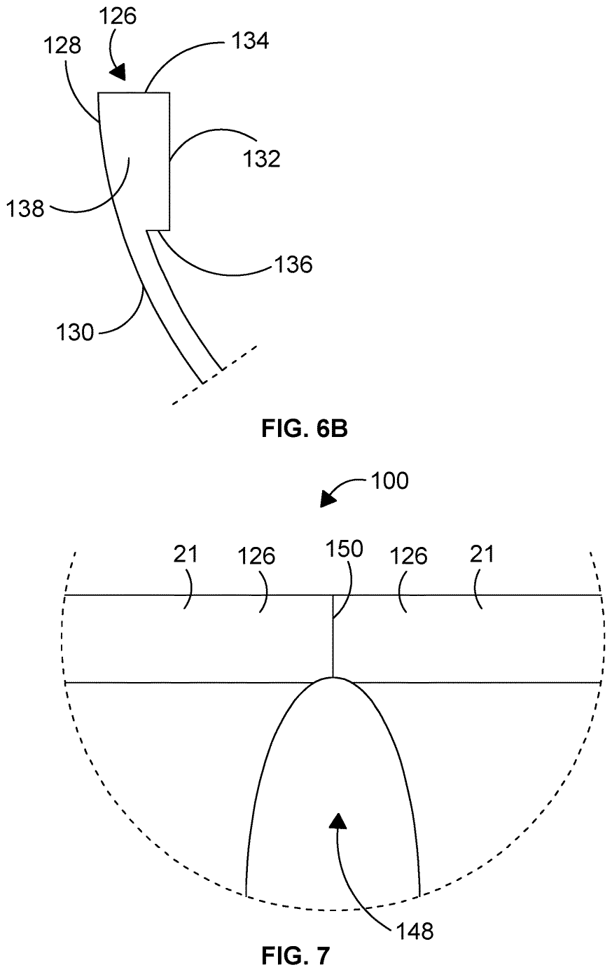

Referring to FIG. 6a, a variant for the adaptor 100 is provided. The FIGS. 5b and 5c provides four arm elements 120 which offer high levels of flexibility radially outward and inward. The circumferential extent of the abutment elements 21 of the arm elements 120 impact upon the contact area between the abutment elements 21 and the abutment locations 407 of the liner 5. The FIG. 6a variant offers increased contact areas. In this case, the three arm elements 120 have a much more extensive base section 122 which links them to the adaptor base 118, a more extensive intermediate section 124 which is wider than the base section 122 and a still more extensive end section 126. The base section 122 provides the main ability for the arm elements 120 to flex radially outwardly and inwardly as needed. Only a small finger-like gap 148 exists between adjacent arm elements 120.

As a consequence of the far more extensive end section 126, the abutment elements 21 can also have a far greater circumferential extent. The abutment locations 407 on the liner has a matching extent and so a far greater contact area between adaptor 100 and line 5 is provided.

The end sections 126 and the abutment elements 21 have a cross-section, shown in FIG. 6b, which is similar to that in the FIGS. 5b and 5c embodiment. The abutment elements 21 have an external surface part 128 which is a smooth continuation of the external arm surface 130. The external arm surface 130 and the external surface part 128 may have a profile complimentary to the part of the shell that they abut in the assembled state. The attachment elements also have an internal surface 132, a distal surface 134, a proximal surface 136 and side surfaces 138.

As illustrated the arm elements 120 form three substantial fan or petal shaped sections which expand out from the base section 122 and therefore extend around a very substantial part of the circumference, perhaps 70% or more.

The further variant of FIG. 7 shows, in a side view, an arrangement for the periphery of the adaptor 100 where the finger-like gap 148 is closed at the periphery and the end sections 126 of the arms abut one another at junction 150. Thus, the attachment elements 21 extend around the whole of the periphery in this variant.

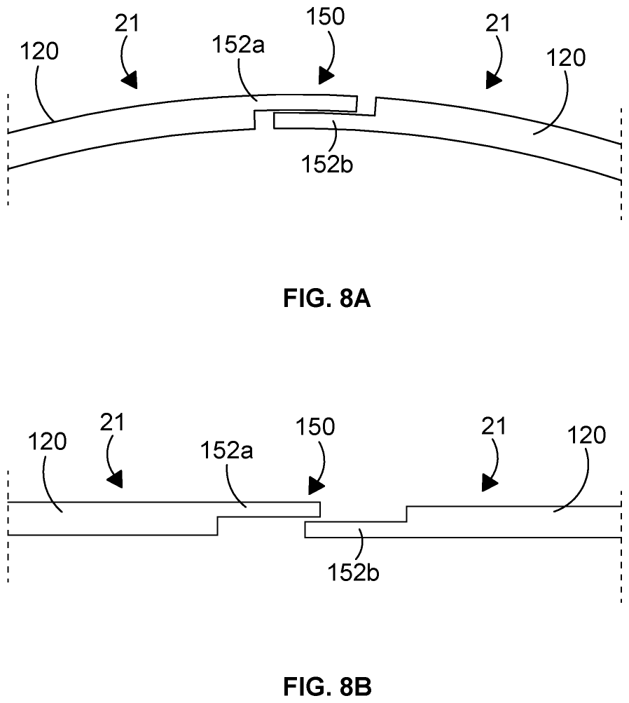

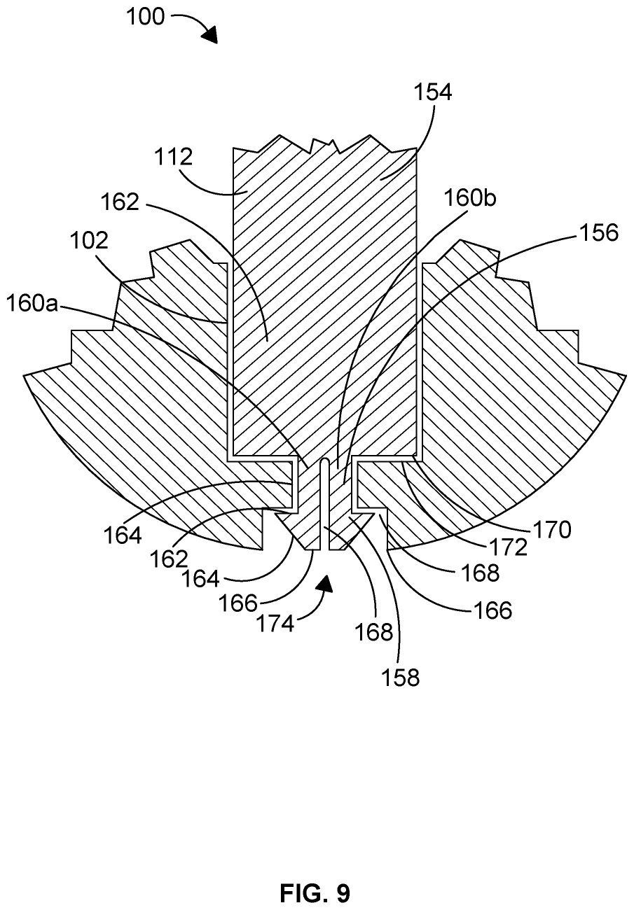

The FIGS. 8a and 8b variant shows, in plan view, an alternative junction 150 between adjacent arm elements 120 in a different variant where the abutment elements 21 extend around the whole of the periphery of the adaptor 100. FIG. 8a shows the adaptor 100 with a first radially extent for the arm elements 120 and FIG. 8b shows the same arm elements 120, but with a greater second radial extent for the arm elements 120. This would be consistent with FIG. 8b being the extent with a larger liner 5 present than in FIG. 8a or consistent with the deformed position for the arm elements 120 when the liner 5 is being inserted, but before the abutment elements 21 move into the abutment locations 7.

In this variant the junction 150 is formed of two overlapping reduced thickness sections 152a and 152b. These allow the flexing to open and increase the diameter of the adaptor 100, but with the reduced thickness sections 152a and 152b still overlapping with one another. One reduced thickness section 152a is provided by an external section and the other reduced thickness section 152b by an internal section.

In the previous embodiments, the adaptor 100 was fixed to the shell 3 using a threaded connection. Other firm forms of connections are possible. In FIG. 9 a variant is illustrated in which the base stem 112 is still provided centrally on the proximal surface 114 of the adaptor 100. The base stem 112 lacks any threaded part 116. Instead, the base stem 112 has a first diameter section 154, followed proximally by a reduced second diameter section 156, followed proximally be a third intermediate diameter section 158. The second diameter section 156 and the third intermediate diameter section 158 are formed by two or more stems, two stems in this illustration, stems 160a and 160b.

Both of the stems 160a, 160b have equivalent, but mirror image profiles. The profiles include a distal facing surface 162, inclined side facing surface 164, proximal facing surface 166 and internal facing surface 168.

In use, when the stems 160a, 160b approach the shell 3, they are positioned in alignment with and then enter an aperture 102 provided in the base 104 of the dished recess 11 of the shell 3. The distal most section 162 of the aperture 102 has a cross-section complimentary to the first diameter section 154 of the adapter 100 and so the stems 160a, 160b can be fully inserted. An intermediate section 164 of the aperture 102 follows the distal most section 162 and has a cross-section through which the stems 160a, 160b can only pass by being deflected toward one another. This is the smallest cross-section for the aperture 102. The inclined side facing surfaces 164 abut the intermediate section 164 first and so encourage the deflection of the stems 160a, 160b towards each other. Proximally beyond the intermediate section 164 of the aperture 102, the proximal most section 166 is reached and this has an intermediate cross-section which allows the stems 160a, 160b to expand outward again. The intermediate section 164 of the aperture and the second diameter section 156 of the adaptor 100 are complimentary to one another. As a result, the position in FIG. 9 is reached for the assembled state. The distal facing surface 162 of the stems 160a, 160b abuts the proximal facing surface 168 of the shell 3. The proximal facing surface 170 of the first diameter section 154 abuts the distal facing surface 172 of the intermediate section 164. A firm mounting of the adaptor 100 relative to the shell 3 is provided.

Whilst the lateral resilience of the stems 160a, 160b can be used, it is possible to introduce an additional element that is driven between the stems 160a, 160b into gap 174 to force them out into position. This type of fixing can be addition to the screw thread mentioned above so as to resist any loosening of the screw thread over time.

The firm fixing of the adaptor 100 to the shell 3 would also be possible through a snap fit or clip type arrangement. Whilst the use of the main apex aperture 102 in the shell 3 is mentioned above, it would be possible to use other through holes, such as other screw fixing holes, distributed over the shell for receipt of these or other fixings.

1. An acetabular component of an orthopaedic joint prosthesis, the acetabular component comprising:
an outer shell for mounting on the acetabulum;
a liner provided at least partially within the outer shell and adapted to provide a contact surface for a femoral component of the orthopaedic joint prosthesis; and
an adaptor provided at least partially within the outer shell and with the liner provided at least partially within the adaptor; wherein:
the acetabular component has an assembled state and in the assembled state relative articulation between the adaptor and the outer shell is prevented.

2. The acetabular component of claim 1, wherein in the assembled state relative articulation between the adapter and the liner is prevented.

3. The acetabular component of claim 1, wherein the adapter has an engaged state with the shell, a plurality of attachment elements engaging with a plurality of attachment locations in the engaged state, at least one of attachment elements or attachment locations being provided on the adapter and the other being provided on the shell and wherein, when the acetabular component is in the assembled state, the shell and adapter are in an engaged state and the adapter and liner are in an engaged state.

4. The acetabular component of claim 1, wherein the liner has an engaged state with the adapter, a plurality of abutment elements engaging with a plurality of abutment locations in the engaged state, at least one of abutment elements or abutment locations being provided on the adapter and the other being provided on the liner.

5. The acetabular component of claim 1, wherein the adapter is compressed by the liner and/or the liner is compressed by the adapter, when the acetabular component is in the assembled state.

6. The acetabular component of claim 1, wherein the outer shell abuts the adapter and the adapter abuts the liner in the assembled state.

7. The acetabular component of claim 1, wherein the adapter has a disengaged state and an engaged state with the shell, together with a transition state between the disengaged state and the engaged state, the adapter having a first configuration in the transition state and a different second configuration in the engaged state.

8. The acetabular component of claim 7, wherein, in the first configuration the attachment elements or attachment locations provided on the adapter are spaced further apart circumferentially and/or radially than in the first configuration.

9. The acetabular component of claim 7, wherein the adapter has a third configuration in the disengaged state and wherein the attachment elements or attachment locations provided on the adapter are spaced further apart circumferentially and/or radially than in the third configuration in the first configuration and/or the second configuration.

10. The acetabular component of claim 1, wherein the adaptor includes a base element and a plurality of arm elements extending from the base element, the abutment elements and/or abutment locations being provided on the arm elements.

11. The acetabular component of claim 10, wherein the arm elements are flexible and flex radially further outward in the transition state compared with the assembled state.

12. The acetabular component of claim 3, according wherein the abutment elements or abutment locations provided on the adapter are provided at the distal periphery of the adapter.

13. The acetabular component of claim 12, wherein the abutment elements and/or abutment locations extend around at least 15% of the distal periphery of the adapter.

14. The acetabular component of claim 13, wherein the abutment elements and/or abutment locations extend around at least 80% of the distal periphery of the adapter.

15. The acetabular component of claim 12, wherein the abutment elements and/or abutment locations extend around the entire distal periphery of the adapter, such as with adjacent abutment elements and/or abutment locations abutting or overlapping with one another.

16. The acetabular component of claim 10, wherein one or more or all of the arm elements include a base section, intermediate section and distal section, the abutment element and/or abutment location being provided in the distal section of each arm.

17. The acetabular component of claim 6, in which the adapter is provided with one or more attachment elements, the attachment elements extending from the base section of the adapter.

18. The acetabular component of claim 1, wherein the adaptor includes as an attachment element an outer shell connection element, and the outer shell includes as an attachment location an adaptor connection location to mount the adapter on the outer shell.

19. The acetabular component of claim 18, wherein the outer shell connection element is a threaded element and the adapter connection element is a threaded element.

20. The acetabular component of claim 18, wherein the outer shell connection element includes one or more distal facing abutment surfaces which abut one or more proximal facing abutment surfaces provided by the adapter connection element.

21-30. (canceled).
